(19) United States
(12) Patent Application Publication
(10) Pub. No.: US 2010/0210965 A1
(43) Pub. Date: Aug. 19, 2010

(54) APPARATUS AND METHOD FOR THE DETECTION OF NEUROMUSCULAR SIGNALS

(76) Inventors: Shai N. Gozani, Brookline, MA (US); Atreyee Gupta, Acton, MA (US); Charles Fendrock, Sudbury, MA (US)

Correspondence Address: Mark J. Pandiscio;Pandiscio & Pandiscio, P.C., 470 Totten Pond Road, Waltham, MA, 02451, US

(21) Appl. No.: 12/371,230

(22) Filed: Feb. 13, 2009

(51) Int. Cl. A61B 5/0488

(52) U.S. Cl. 600/546; 600/554

(57) ABSTRACT

A multi-electrode assembly for use in neuromuscular testing, the multi-electrode assembly comprising:

a substrate;

at least one stimulation electrode carried by the substrate;

at least one detection electrode carried by the substrate;

a connector carried by the substrate, the connector being configured for connection with testing electronics; and

a plurality of traces for electrically connecting the at least one stimulation electrode and the at least one detection electrode to the connector;

wherein the substrate is flexible so that the at least one stimulation electrode and the at least one detection electrode are independently positionable on a patient.

### FIELD OF THE INVENTION

This invention relates to an improved apparatus and method for the assessment of neuromuscular function. More particularly, this invention relates to an improved apparatus and method for the detection of neuromuscular signals which are indicative of neuromuscular function.

### BACKGROUND OF THE INVENTION

There are many clinical and non-clinical situations which call for a rapid, reliable and low-cost assessment of neuromuscular function. This assessment of neuromuscular function is generally conducted in order to identify and assess the nature and extent of neuromuscular disorders.

### Neuromuscular Disorders

Neuromuscular disorders are relatively common and typically relate to pathologies of the peripheral nerves and muscles. Many are well known to the general public.

By way of example but not limitation, Carpal Tunnel Syndrome (CTS) is a common form of neuromuscular disease. CTS arises from compression of the median nerve in the region where the median nerve traverses the wrist. CTS typically causes discomfort and/or loss of sensation in the hand and, in severe cases, may compromise use of the hand. Highly repetitive wrist movements are believed to be one cause of CTS.

By way of further example but not limitation, Diabetic Peripheral Neuropathy (DPN) is another common form of neuromuscular disease. DPN generally arises as a complication from diabetes. More particularly, diabetes can cause damage to small blood vessels over time, which can in turn lead to damage to peripheral nerves. This peripheral nerve damage can cause tingling, numbness, unusual sensations, pain, etc. The areas of the body most commonly affected by DPN are the feet and legs.

Other common medical conditions (e.g., rheumatoid arthritis, cancer, etc.) are also associated with neuromuscular disease.

### Peripheral Nerves And Muscles

The peripheral nerves consist of motor nerves and sensory nerves. Motor nerves control muscles and sensory nerves carry sensory information from the periphery of the body into the central nervous system.

Motor nerve function is generally assessed by electrically stimulating a nerve and then measuring the response of the muscle innervated by that nerve. The muscle response is detected by measuring the myoelectric potential generated by the muscle in response to the stimulus applied to the nerve.

Sensory nerve function is generally assessed by electrically stimulating a sensory nerve at a first point along the nerve (i.e., the “stimulation site”) and then measuring the bioelectrical response of the same nerve at a second point along the nerve (i.e., the “detection site”), with the detection site being located a known distance from the stimulation site.

With respect to neuromuscular function, one indication of the physiological state of the nerve is provided by the time delay occurring between the application of the stimulus to the nerve and the detection of the resulting bioelectrical response, where the resulting bioelectrical response may be located in a muscle (i.e., in the case of a motor nerve) or in another part of the nerve (i.e., in the case of a sensory nerve). More particularly, in the case of neuromuscular disease affecting motor nerves, if the nerve is damaged, conduction of the signal via the nerve to the muscle, and hence detection of the muscle's resulting myoelectrical response, will be slower than in a healthy nerve. Correspondingly, in the case of disease affecting sensory nerves, if the nerve is damaged, conduction of the signal from the stimulation site to the detection site will be slower than in a healthy nerve. Thus, an abnormally long delay between the application of a stimulus to the nerve and the detection of a muscle or sensory nerve response generally indicates impaired neuromuscular function.

Skin temperature also affects the time delay occurring between when the stimulus is applied to the nerve and when the response is detected in the muscle (in the case of a motor nerve) or distal nerve section (in the case of a sensory nerve). In general, a lower skin temperature results in a longer delay than a warmer skin temperature.

### Signal-Detecting Electrodes

The neuromuscular testing described above is generally performed with either (i) individually-placed, pre-gelled disposable electrodes, or (ii) pre-configured, nerve-specific, multi-electrode assemblies. In either case, the active electrode areas are first placed against the surface of the skin at specific anatomical sites, and then at least one of the electrodes is used to stimulate the nerve and at least another one of the electrodes is used to detect the resulting neuromuscular signal.

### Individually-Placed Electrodes for Assessing Neuromuscular Function

Individually-placed, pre-gelled disposable electrodes applied to the skin are used extensively in neuromuscular testing. A typical procedure utilizes a plurality of these individually-placed, pre-gelled disposable electrodes applied to the appropriate anatomical areas, with certain individually-placed electrodes being used to electrically stimulate the nerve of interest and other individually-placed electrodes being used to detect the evoked neuromuscular response. A minimum of five electrodes are typically used for a neuromuscular motor or sensory nerve test. Sensory nerve testing is often performed on the fingers of the patient using ring electrodes, i.e., long electrodes that wrap around the circumference of the patient's finger.

Individually-placed, pre-gelled disposable electrodes are popular for neuromuscular testing, particularly among sophisticated healthcare providers (e.g., neurologists), due largely to their versatility—the healthcare provider can use the individually-placed, pre-gelled disposable electrodes with substantially any motor nerve and/or sensory nerve, setting the electrode configuration according to the nerve, its anatomical disposition and/or the individual preferences of the healthcare provider. However, individually-placed, pre-gelled disposable electrodes also suffer from a number of drawbacks.

For one thing, the individually-placed electrodes are not labeled in any way with the location on the body to which the electrodes should be applied. This is, of course, due to the fact that the individually-placed, pre-gelled disposable electrodes are deliberately intended to be placed at a wide range of different locations on the body. However, this can also result in errors in electrode placement, particularly with healthcare providers who may have less training and/or less experience with neuromuscular testing.

For another thing, each electrode has a separate wire to electrically connect the electrode to the testing electronics. Inasmuch as multiple electrodes are used in a typical neuromuscular test, this requires that the healthcare provider make multiple electrical connections during set-up and typically results in a tangle of wires at the test site. This can be time-consuming and inconvenient for the healthcare provider, and also raises the possibility of connection errors when connecting the electrodes to the testing electronics, particularly among healthcare providers who may have less training and/or less experience with neuromuscular testing. Furthermore, the loose tangle of wires may also pick up significant electromagnetic interference (EMI) from the lights, computers and/or other electrical equipment commonly found in a medical office.

In addition to the foregoing, as noted above, it is well known that skin temperature also has an effect on the results of a neurological test, i.e., a colder nerve has a lower conduction velocity than a warmer nerve. Therefore, when using individually-placed electrodes, if the healthcare provider wants to compensate for temperature effects, then a separate skin temperature measurement must be made for different locations on the skin. This may be done by either (i) using individually-placed temperature sensors (which adds to the set-up time, the tangle of wires at the test site, possible connection error, etc.), or (ii) adding temperature sensors to each of the individually-placed electrodes (which adds to cost, complicates connection requirements, etc.).

### Pre-Configured, Nerve-Specific, Multi-Electrode Assemblies for Assessing Neuromuscular Function

Electrodiagnostic devices utilizing pre-configured, nerve-specific, multi-electrode asssemblies have been introduced into the marketplace to assess neuromuscular function in physician offices. These electrodiagnostic devices are often highly automated and are frequently intended to be used by healthcare providers lacking specialized neurophysiology training.

More particularly, U.S. Pat. No. 5,976,094 to Gozani discloses one such electrodiagnostic device that is successfully used thousands of times a year to make diagnostic assessments of peripheral nerves. The Gozani device is intended to use a pre-configured, nerve-specific, multi-electrode assembly when assessing neuromuscular function. More particularly, the pre-configured, nerve-specific, multi-electrode assembly of the Gozani device comprises at least one proximal stimulation electrode that is set in a fixed geometric relationship to at least one distal detection electrode. The stimulation electrode applies an electrical stimulation to a nerve, and the detection electrode detects the resulting bioelectric potential (i.e., signal) that is evoked downstream of the stimulation site, either in the muscle innervated by that nerve (in the case of the assessment of a motor nerve) or in the nerve itself (in the case of the assessment of a sensory nerve). The pre-configured, nerve-specific, multi-electrode assembly of the Gozani device is intended to be quickly, easily and correctly positioned against the patient's anatomy using well-known anatomical landmarks, even by healthcare providers lacking specialized neurophysiology training.

In order to maintain the at least one stimulation electrode in a fixed geometric relationship to the at least one detection electrode, the pre-configured, nerve-specific, multi-electrode assembly of the Gozani device comprises a semi-rigid substrate to which the various electrodes are mounted. This semi-rigid substrate is rigid enough to hold the various electrodes in a fixed configuration relative to one another.

The pre-configured, nerve-specific, multi-electrode assembly of the Gozani device significantly improves the speed and accuracy with which a neurological test can be performed, particularly with healthcare providers lacking specialized neurophysiology training. However, the pre-configured, nerve-specific, multi-electrode assembly of the Gozani device is designed to perform a test on one specific nerve, usually on only one side (left or right) of the body. Performing a test on the same nerve on the other side of the body for a bilateral test, or performing a test on an altogether different nerve, requires the use of a different pre-configured, nerve-specific, multi-electrode assembly. This need to use different pre-configured, nerve-specific, multi-electrode assemblies for different nerves and/or different anatomical locations increases the cost of neurological testing where multiple nerve locations must be tested, since then multiple pre-configured, nerve-specific, multi-electrode assemblies must be used in the course of the complete test. Furthermore, the need to use different pre-configured, nerve-specific, multi-electrode assemblies for different nerves and/or different anatomical locations requires that the healthcare provider maintain an adequate inventory of different pre-configured, nerve-specific, multi-electrode assemblies, i.e., at least one pre-configured, nerve-specific, multi-electrode assembly for each different nerve and/or each different anatomical location which is to be tested. This can present a burden to small healthcare providers, e.g., a small medical practice. Also, the pre-configured, nerve-specific, multi-electrode assemblies of the Gozani device are intended to be single-use only, which can increase the cost of a neurological procedure where the procedure involves testing multiple nerves and/or multiple nerve locations.

### OBJECTS OF THE INVENTION

As a result, one object of the present invention is to provide a new multi-electrode assembly which reduces the number of electrical connections which must be made when connecting the electrodes to the testing electronics, whereby to eliminate the tangle of wires, possible connection errors and time delays associated with some prior art approaches.

Another object of the present invention is to provide a new multi-electrode assembly which minimizes errors in electrode placement when positioning the electrodes on the patient.

Another object of the present invention is to provide a new multi-electrode assembly which reduces the effects of EMI on a neuromuscular test.

Another object of the present invention is to provide a new multi-electrode assembly which includes an integrated means to compensate for skin temperature.

Another object of the present invention is to provide a new multi-electrode assembly which reduces the time necessary to conduct a neuromuscular test, in order to increase convenience for the patient and improve the efficiency of the healthcare provider.

Another object of the present invention is to provide a new multi-electrode electrode assembly which can be used to perform a neuromuscular test on a wide range of different nerves and/or different anatomical locations, so as to reduce the number of electrodes which may be needed to conduct a neuromuscular test, in order to help reduce the cost of conducting a neuromuscular test.

Another object of the present invention is to provide a new multi-electrode assembly which can be used to perform a neuromuscular test on a wide range of different nerves and/or different anatomical locations, so that the number of types of test electrodes that must be inventoried by the healthcare provider is greatly reduced.

### SUMMARY OF THE INVENTION

These and other objects of the present invention are addressed by the provision and use of a new multi-electrode assembly for use in neuromuscular testing, wherein the new multi-electrode assembly comprises a substrate to which is mounted at least one stimulation electrode and at least one detection electrode, and further wherein the substrate is flexible so as to permit the healthcare provider to set the electrodes in a variety of different configurations in order to permit the multi-electrode assembly to be used to perform neuromuscular tests on a wide range of different nerves and/or a wide range of anatomical locations. Preferably, the substrate provides guidance to the healthcare provider to facilitate correct anatomical placement of the electrodes, and the at least one stimulation electrode and the at least one detection electrode are electrically connected via traces to a single connector in order to simplify proper connection to the testing electronics. Furthermore, the new multi-electrode assembly preferably includes an integrated means to compensate for skin temperature.

In one preferred form of the invention, there is provided a multi-electrode assembly for use in neuromuscular testing, the multi-electrode assembly comprising:

a substrate;

at least one stimulation electrode carried by the substrate;

at least one detection electrode carried by the substrate;

a connector carried by the substrate, the connector being configured for connection with testing electronics; and

a plurality of traces for electrically connecting the at least one stimulation electrode and the at least one detection electrode to the connector;

wherein the substrate is flexible so that the at least one stimulation electrode and the at least one detection electrode are independently positionable on a patient.

In another preferred form of the invention, there is provided a method for conducting neuromuscular testing on a patient, the method comprising:

providing a multi-electrode assembly comprising:

a substrate;

at least one stimulation electrode carried by the substrate;

at least one detection electrode carried by the substrate;

a connector carried by the substrate, the connector being configured for connection with testing electronics; and

a plurality of traces for electrically connecting the at least one stimulation electrode and the at least one detection electrode to the connector;

wherein the substrate is flexible so that the at least one stimulation electrode and the at least one detection electrode are independently positionable on a patient;

positioning the multi-electrode assembly on a patient;

connecting the connector to testing electronics; and

performing a neuromuscular test on the patient.

In another preferred form of the invention, there is provided a multi-electrode assembly for use in neuromuscular testing, the multi-electrode assembly comprising:

a substrate comprising first, second, third, fourth, fifth and sixth islands, and first, second, third, fourth and fifth bridges for connecting the first and second islands, the second and third islands, the first and fourth islands, the fourth and fifth islands and the fifth and sixth islands, respectively;

a first stimulation electrode carried by the first island, and a second stimulation electrode carried by the first island;

a first active detection electrode carried by the second island;

an inactive detection electrode carried by the third island;

a reference electrode carried by the fourth island;

a second active detection electrode carried by the fifth island;

a third active detection electrode carried by the sixth island;

a connector carried by the substrate, the connector being configured for connection with testing electronics; and

a plurality of traces for electrically connecting the first and second stimulation electrodes, first active detection electrode, inactive detection electrode, a reference electrode, third active detection electrode and fourth active detection electrode to the connector;

wherein the substrate is flexible so that the first and second stimulation electrodes, first active detection electrode, inactive detection electrode, reference electrode, third active detection electrode and fourth active detection electrode are independently positionable on a patient.

In another preferred form of the invention, there is provided a method for conducting neuromuscular testing on a patient, the method comprising:

providing a multi-electrode assembly comprising:
a substrate comprising first, second, third, fourth, fifth and sixth islands, and first, second, third, fourth and fifth bridges for connecting the first and second islands, the second and third islands, the first and fourth islands, the fourth and fifth islands and the fifth and sixth islands, respectively;
a first stimulation electrode carried by the first island, and a second stimulation electrode carried by the first island;
a first active detection electrode carried by the second island;
an inactive detection electrode carried by the third island;
a reference electrode carried by the fourth island;
a second active detection electrode carried by the fifth island;
a third active detection electrode carried by the sixth island;
a connector carried by the substrate, the connector being configured for connection with testing electronics; and
a plurality of traces for electrically connecting the first and second stimulation electrodes, first active detection electrode, inactive detection electrode, a reference electrode, third active detection electrode and fourth active detection electrode to the connector;
wherein the substrate is flexible so that the first and second stimulation electrodes, first active detection electrode, inactive detection electrode, reference electrode, third active detection electrode and fourth active detection electrode are independently positionable on a patient;

positioning the multi-electrode assembly on a patient;

connecting the connector to testing electronics; and

performing a neuromuscular test on the patient.

In another preferred form of the invention, there is provided a multi-electrode assembly for use in neuromuscular testing, the multi-electrode assembly comprising:

a substrate comprising first, second, third and fourth islands, and first, second and third bridges for connecting the first and second islands, the second and third islands and the third and fourth islands, respectively;

a first stimulation electrode carried by the first island;

a first active detection electrode carried by the third island;

an inactive detection electrode carried on the fourth island;

a second active detection electrode carried by the third island;

a reference electrode carried by the second island;

a connector carried by the substrate, the connector being configured for connection with testing electronics; and

a plurality of traces for electrically connecting the first stimulation electrode, first active detection electrode, inactive detection electrode, second active detection electrode and reference electrode to the connector;

wherein the substrate is flexible so that the first stimulation electrode, first active detection electrode, inactive detection electrode, second active detection electrode and reference electrode are independently positionable on a patient.

In another preferred form of the invention, there is provided a method for conducting neuromuscular testing on a patient, the method comprising:

providing a multi-electrode assembly comprising:
a substrate comprising first, second, third and fourth islands, and first, second and third bridges for connecting the first and second islands, the second and third islands and the third and fourth islands, respectively;
a first stimulation electrode carried by the first island;
a first active detection electrode carried by the third island;
an inactive detection electrode carried on the fourth island;
a second active detection electrode carried by the third island;
a reference electrode carried by the second island;
a connector carried by the substrate, the connector being configured for connection with testing electronics; and
a plurality of traces for electrically connecting the first stimulation electrode, first active detection electrode, inactive detection electrode, second active detection electrode and reference electrode to the connector;
wherein the substrate is flexible so that the first stimulation electrode, first active detection electrode, inactive detection electrode, second active detection electrode and reference electrode are independently positionable on a patient;

positioning the multi-electrode assembly on a patient;

connecting the connector to testing electronics; and

performing a neuromuscular test on the patient.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIG. 1 is a perspective view of a new multi-electrode assembly formed in accordance with the present invention;

FIG. 2 is a front view of the multi-electrode assembly shown in FIG. 1, taken from the frame of reference of FIG. 1;

FIG. 3 is a rear view of the multi-electrode assembly shown in FIG. 1, taken from the frame of reference of FIG. 1;

FIG. 4 is a left side view, in elevation, of the multi-electrode assembly shown in FIG. 1, taken from the frame of reference of FIG. 2;

FIG. 5 is a right side view, in elevation, of the multi-electrode assembly shown in FIG. 1, taken from the frame of reference of FIG. 2;

FIG. 6 is a top end view, in elevation, of the multi-electrode assembly shown in FIG. 1, taken from the frame of reference of FIG. 2;

FIG. 7 is a bottom end view, in elevation, of the multi-electrode assembly shown in FIG. 1, taken from the frame of reference of FIG. 2;

FIG. 8 is a perspective view of another multi-electrode assembly formed in accordance with the present invention;

FIG. 9 is a front view of the multi-electrode assembly shown in FIG. 8, taken from the frame of reference of FIG. 8;

FIG. 10 is a rear view of the multi-electrode assembly shown in FIG. 8, taken from the frame of reference of FIG. 8;

FIG. 11 is a left side view, in elevation, of the multi-electrode assembly shown in FIG. 8, taken from the frame of reference of FIG. 9;

FIG. 12 is a right side view, in elevation, of the multi-electrode assembly shown in FIG. 8, taken from the frame of reference of FIG. 9;

FIG. 13 is a top end view, in elevation, of the multi-electrode assembly shown in FIG. 8, taken from the frame of reference of FIG. 9; and

FIG. 14 is a bottom end view, in elevation, of the multi-electrode assembly shown in FIG. 8, taken from the frame of reference of FIG. 9.

### DETAILED DESCRIPTION OF THE PREFERRED EMBODIMENTS

### The New Multi-Electrode Assembly in General

As noted above, the present invention comprises the provision and use of a new multi-electrode assembly for use in neuromuscular testing, wherein the new multi-electrode assembly comprises a substrate to which is mounted at least one stimulation electrode and at least one detection electrode, and further wherein the substrate is flexible so as to permit the healthcare provider to set the electrodes in a variety of different configurations in order to permit the multi-electrode assembly to be used to perform neuromuscular tests on a wide range of different nerves and/or a wide range of anatomical locations. Preferably, the substrate provides guidance to the healthcare provider to facilitate correct anatomical placement of the electrodes, and the at least one stimulation electrode and the at least one detection electrode are electrically connected via traces to a single connector in order to simplify proper connection to the testing electronics. Furthermore, the new multi-electrode assembly preferably includes an integrated means to compensate for skin temperature.

Because the multi-electrode assembly is intended to be used to perform neuromuscular tests on a wide range of different nerves and/or a wide range of anatomical locations, and because the multi-electrode assembly is intended to be used on patients of varying anatomical sizes, the multi-electrode assembly is sized so as to be able to reach the desired points on the patient for the desired nerves and anatomical locations across the expected range of patient sizes.

Furthermore, in some cases, the multi-electrode assembly may need to be positioned on the body and then used for both a motor nerve test and a sensory nerve test. In other cases, the multi-electrode assembly may need to be positioned on the body and then used for either a motor nerve test or a sensory nerve test, but not both (without repositioning). This consideration leads to the provision of two different multi-electrode assemblies.

### The Multi-Electrode Assembly which may be Positioned on the Body and then used for Both a Motor Nerve Test and a Sensory Nerve Test

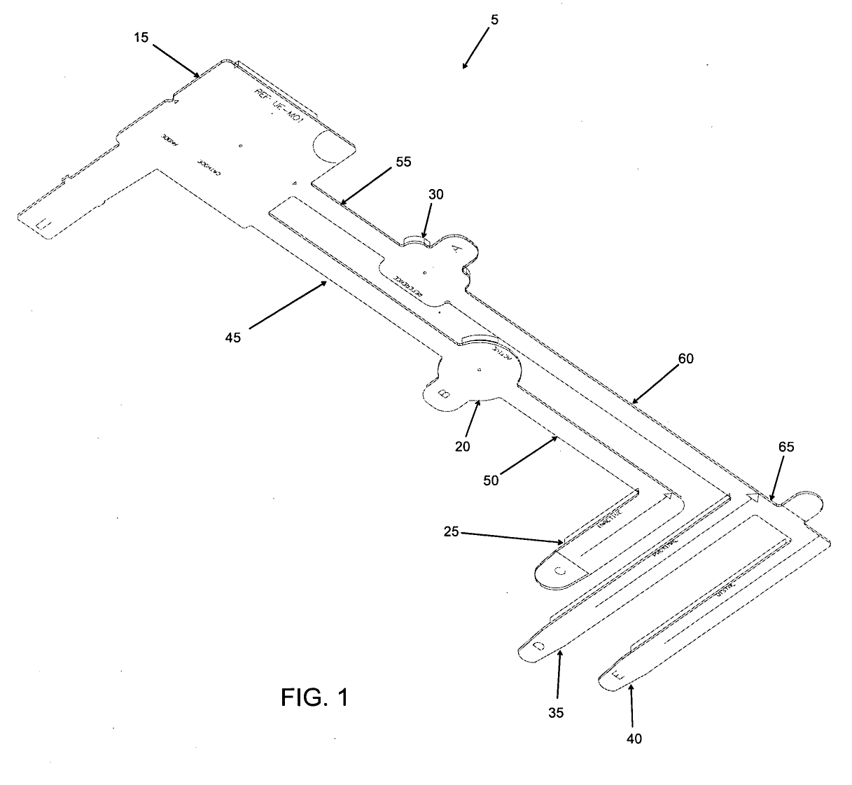

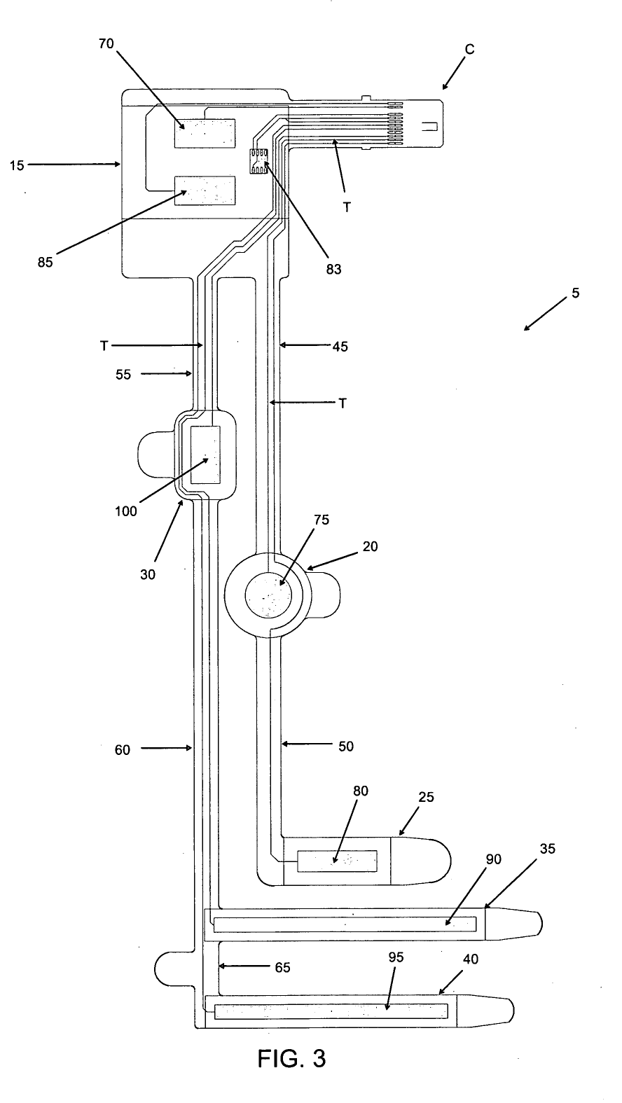

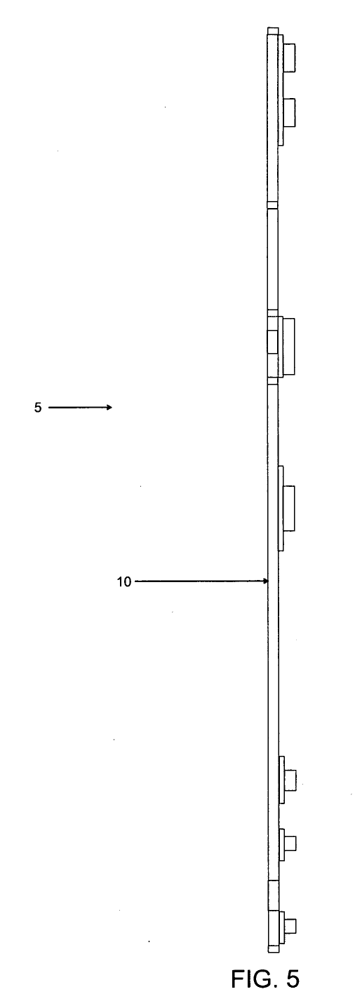

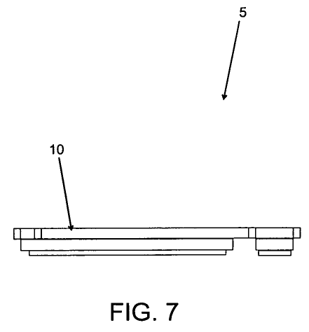

Looking first to FIGS. 1-7, there is shown a multi-electrode assembly 5 formed in accordance with the present invention. Multi-electrode assembly 5 is designed to be positioned on the body and then used for both a motor nerve test and a sensory nerve test.

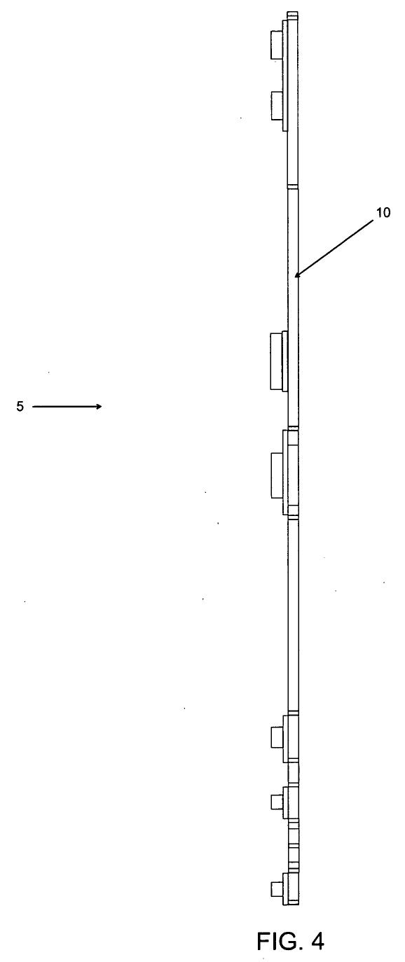

To this end, multi-electrode assembly 5 generally comprises a substrate 10 (FIGS. 4-7). Substrate 10 comprises islands 15, 20, 25, 30, 35 and 40. Islands 15 and 20 are connected by a bridge 45; islands 20 and 25 are connected by a bridge 50; islands 15 and 30 are connected by a bridge 55; islands 30 and 35 are connected by a bridge 60; and islands 35 and 40 are connected by a bridge 65.

Substrate 10 is formed out of a flexible material. Inasmuch as bridges 45, 50, 55, 60 and 65 are characterized by elongated geometries, these bridges have a highly flexible character. To the extent that islands 15, 20, 25, 30, 35 and 40 (and particularly islands 15, 20 and 30) have less elongated structures, these islands may have a somewhat less flexible character. However, islands 35 and 40 are preferably formed with a flexibility such that they can be used as “ring electrodes” that wrap around the circumference of a finger of a patient. In essence, bridges 45, 50, 55, 60 and 65 are sufficiently flexible to permit the healthcare provider to independently position islands 15, 20, 25, 30, 35 and 40 on the patient in precisely the manner desired by the healthcare provider, and islands 15, 20, 25, 30, 35 and 40 have sufficient structural integrity to permit the healthcare provider to easily handle and manipulate the islands, in order to facilitate their accurate placement on the patient.

In order to provide multi-electrode assembly 5 with the capacity to perform a motor nerve test, stimulation electrodes 70 and 85 are provided on island 15, an active detection electrode 75 is provided on island 20, an inactive detection electrode 80 is provided on island 25, and a reference electrode 100 is provided on island 30. Traces T electrically connect stimulation electrodes 70 and 85, active detection electrode 75, inactive detection electrode 80 and reference electrode 100 to a connector C. Preferably, a temperature sensor 83 is also provided on island 15 and is connected to connector C via a trace T.

Stimulation electrodes 70 and 85 are intended to be placed over a nerve, active detection electrode 75 placed over a muscle innervated by that nerve, inactive detection electrode 80 placed over another portion of the anatomy, reference electrode 100 is place over another portion of the anatomy, temperature sensor 83 placed against the skin and connector C connected to the testing electronics. Stimulation electrodes 70 and 85 are used to stimulate a nerve and active detection electrode 75 is used to detect the response of a muscle innervated by that nerve, with inactive detection electrode 80 being used to eliminate noise in the signal, with reference electrode 100 being used as the zero volt reference of the signal, and with temperature sensor 83 being used to compensate for skin temperature.

In order to provide multi-electrode assembly 5 with the capacity to perform a sensory nerve test, stimulation electrodes 70 and 85 are provided on island 15, a first active detection electrode 90 is provided on island 35, a second active electrode 95 is provided on island 40 and reference electrode 100 is provided on island 30. Traces T electrically connect stimulation electrodes 70 and 85, first active detection electrode 90, second active detection electrode 95 and reference electrode 100 to connector C. As noted above, temperature sensor 83 is preferably provided on island 15 and is connected to connector C via a trace T.

Stimulation electrodes 70 and 85 are intended to be placed over a portion of a nerve, first active detection electrode 90 placed over a proximal sensory portion of that same nerve, second active detection electrode 95 placed over a distal sensory portion of that same nerve, reference electrode 100 placed over another portion of the anatomy, temperature sensor 83 placed on the skin and connector C is connected to the testing electronics. Stimulation electrodes 70 and 85 are used to stimulate the nerve, and both of first active detection electrode 90 and second active detection electrode 95 are used to detect the resulting signal in the distal portion of that nerve, with reference electrode 100 being used as the zero volt reference of the signal and temperature sensor 83 being used to compensate for skin temperature.

Significantly, inasmuch as all of the active components of multi-electrode assembly 5 are formed on a single substrate, the components can be conveniently advanced to the body as a single unit and connected to the testing electronics via a single connector, thereby facilitating speed of deployment and accuracy of connection. Furthermore, inasmuch as each of the active components of multi-electrode assembly 5 is securely mounted to an easily manipulated island, and inasmuch as the islands are connected together via highly flexible bridges, the healthcare provider is able to easily place the active components on the patient's anatomy with a wide range of choices. As a result, a single multi-electrode assembly 5 can be used to test a wide range of different nerves, at a wide range of different anatomical locations, for a wide range of anatomical sizes.

Furthermore, inasmuch as multi-electrode assembly 5 includes all of the elements needed to perform a motor nerve test (e.g., stimulation electrodes 70 and 85, active detection electrode 75, inactive detection electrode 80, and reference electrode 100) and also includes all of the elements needed to perform a sensory nerve test (e.g., stimulation electrode 85, first active detection electrode 90, second active detection electrode 95 and reference electrode 100), with all of the active elements being electrically connected to single connector C, multi-electrode assembly 5 may be positioned on the body and then used with both a motor nerve test and a sensory nerve test. In other words, in some cases, multi-electrode assembly 5 may be positioned on the patient so that stimulation electrodes 70 and 85 stimulate both motor and sensory nerves. In this respect it should also be appreciated that once multi-electrode assembly 5 has been precisely secured to the patient and connector C connected to the testing electronics, the healthcare provider can conduct one or both of the motor nerve test and the sensory nerve test in any order desired, or even simultaneously if desired.

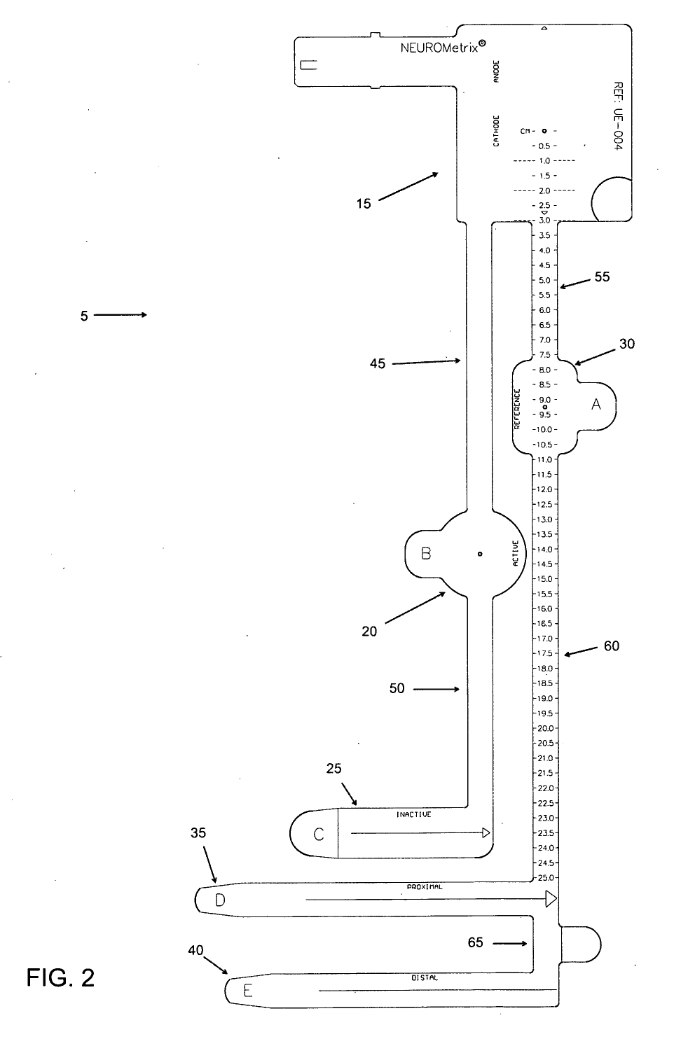

As seen in FIG. 2, the front side of substrate 10 is preferably labeled so as to show the location of the various elements. This labeling helps the healthcare provider avoid errors when applying the electrodes to the patient. Also printed on the front side of substrate 10 is a measurement scale to be used by the healthcare provider in accurately determining the distance between the stimulation site and a detection site when performing a sensory or motor nerve assessment.

It should be appreciated that traces T are disposed on substrate 10 so as to be closely spaced and of short length, whereby to reduce the electrical noise effects of external EMI. This is because the short traces and small cross-sectional areas between the traces reduce induced electromagnetic noise signals.

### The Multi-Electrode Assembly which may be Positioned on the Body and then used for Either a Motor Nerve Test or a Sensory Nerve Test, But Not Both (Without Repositioning)

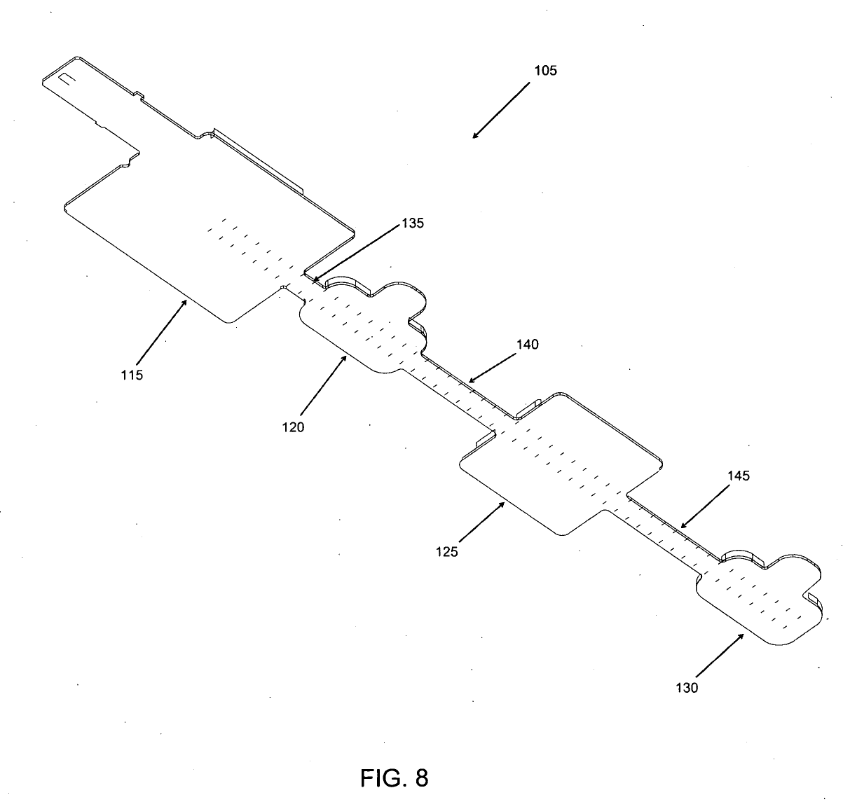

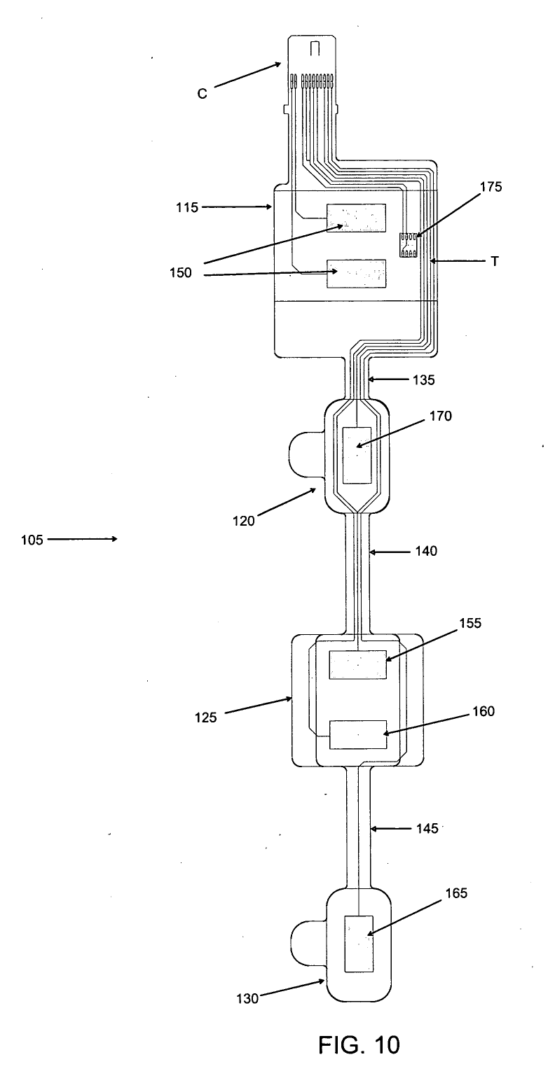

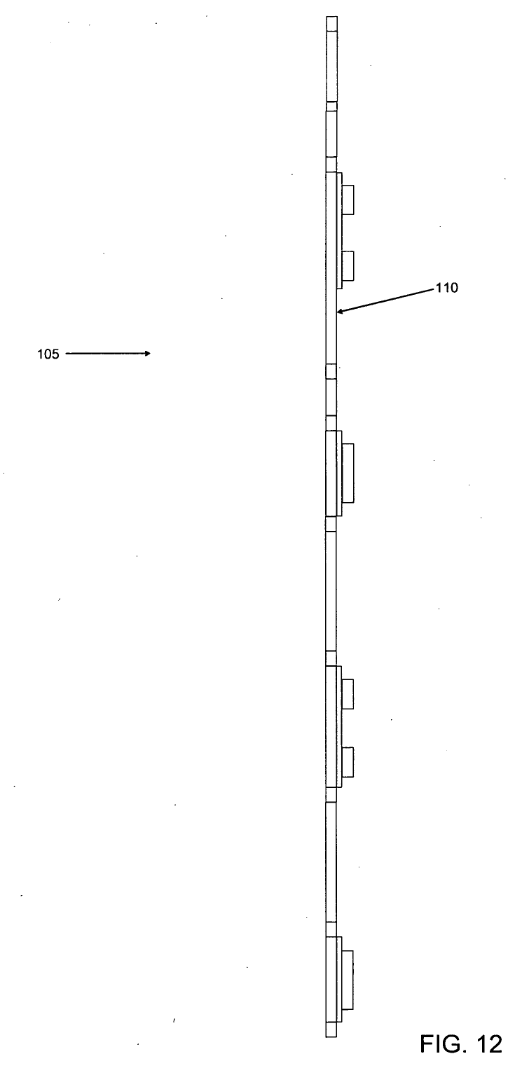

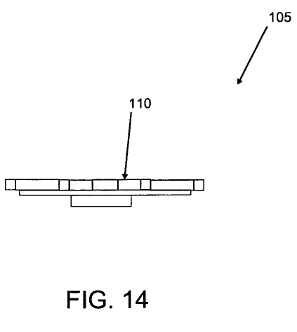

Looking next at FIGS. 8-14, there is shown a multi-electrode assembly 105 also formed in accordance with the present invention. Multi-electrode assembly 105 is designed to be positioned on the body and then used for either a motor nerve test or a sensory nerve test, but not both (without repositioning).

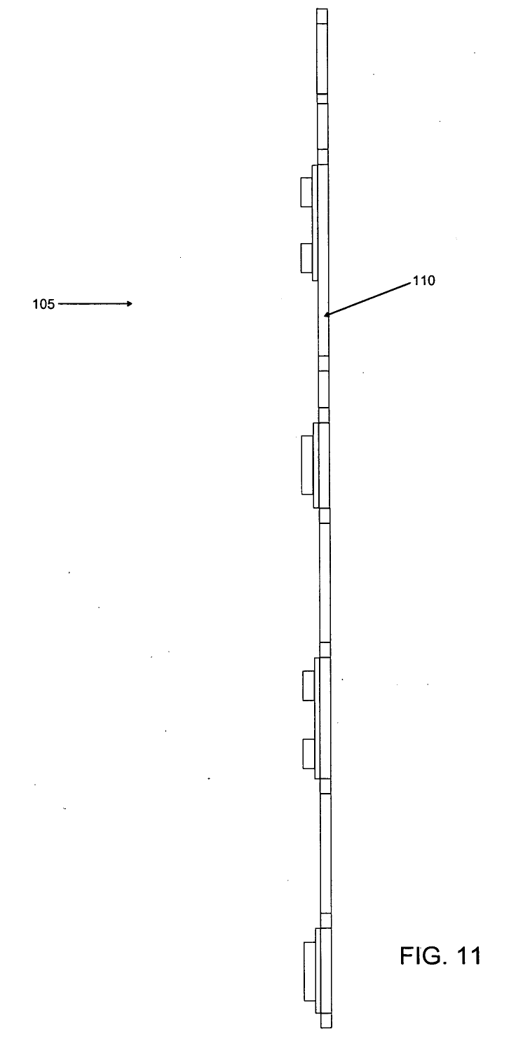

To this end, multi-electrode assembly 105 generally comprises a substrate 110 (FIGS. 11-14). Substrate 110 comprises islands 115, 120, 125 and 130. Islands 115 and 120 are connected by a bridge 135; islands 120 and 125 are connected by a bridge 140; and islands 125 and 130 are connected by a bridge 145.

Substrate 110 is formed out of a flexible material. Inasmuch as bridges 135, 140 and 145 are characterized by elongated geometries, these bridges have a highly flexible character. To the extent that islands 115, 120, 125 and 130 have less elongated structures, these islands may have a somewhat less flexible character. In essence, bridges 135, 140 and 145 are sufficiently flexible to permit the healthcare provider to independently position islands 115, 120, 125 and 130 on the patient in precisely the manner desired by the healthcare provider, and islands 115, 120, 125 and 130 have sufficient structural integrity to permit the healthcare provider to easily handle and manipulate the islands, in order to facilitate their accurate placement on the patient.

In order to provide multi-electrode assembly 105 with the capacity to perform a motor nerve test or a sensory nerve test, one or more stimulation electrodes 150 are provided on island 115, a first active detection electrode 155 is provided on island 125, a second active detection electrode 160 is provided on island 125, an inactive detection electrode 165 is provided on island 130 and a reference electrode 170 is provided on island 120. Traces T electrically connect the one or more stimulation electrodes 150, first active detection electrode 155, second active detection electrode 160, inactive detection electrode 165 and reference electrode 170 to connector C. Preferably, a temperature sensor 175 is provided on island 115 and connected to connector C via a trace T.

For testing a motor nerve, the one or more stimulation electrodes 150 are placed over a nerve, first active detection electrode 155 is placed over a muscle innervated by that nerve, inactive detection electrode 165 is placed over another portion of the anatomy, reference electrode 170 is placed over another portion of the anatomy, temperature sensor 175 is placed against the skin and connector C is connected to the testing electronics. Then, one or more of the stimulation electrodes 150 are used to stimulate a nerve and first active detection electrode 155 is used to detect the response of a muscle innervated by that nerve, with inactive detection electrode 165 being used to eliminate noise in the signal, with reference electrode 170 being used to provide zero volt reference in the signal, and with temperature sensor 175 being used to compensate for skin temperature.

For testing a sensory nerve, the one or more stimulation electrodes 150 are placed over a portion of a nerve, first active detection electrode 155 is placed over a proximal portion of that same nerve, second active detection electrode 160 is placed over a distal portion of that same nerve, reference electrode 170 is placed over another portion of the anatomy, temperature sensor 175 is placed on the skin and connector C is connected to the testing electronics. Then, one or more of the stimulation electrodes 150 are used to stimulate a nerve, and one or both of first active detection electrode 155 and second active detection electrode 160 are used to detect the signal in the sensory portion of that nerve, with reference electrode 170 being used to provide zero volt reference in the signal and temperature sensor 175 being used to compensate for skin temperature.

Significantly, inasmuch as all of the active components of multi-electrode assembly 105 are formed on a single substrate, the components can be conveniently advanced to the body as a single unit and connected to the testing electronics via a single connector, thereby facilitating speed of deployment and accuracy of connection. Furthermore, inasmuch as each of the active components of multi-electrode assembly 105 is securely mounted to an easily manipulated island, and inasmuch as the islands are connected together via highly flexible bridges, the healthcare provider is able to easily place the active components on the patient's anatomy with a wide range of choices. As a result, a single multi-electrode assembly 105 can be used to test a wide range of different nerves, at a wide range of different anatomical locations, for a wide range of anatomical sizes.

Furthermore, inasmuch as multi-electrode assembly 105 includes all of the elements needed to perform a motor nerve test (e.g., one or more stimulation electrodes 150, first active detection electrode 155, inactive detection electrode 165 and reference electrode 170) and also includes all of the elements needed to perform a sensory nerve test (e.g., one or more stimulation electrodes 150, first active detection electrode 155, second active electrode 160 and reference electrode 170), with all of the active elements being electrically connected to single connector C, multi-electrode assembly may be positioned on the body and then used for either a motor nerve test or a sensory nerve test.

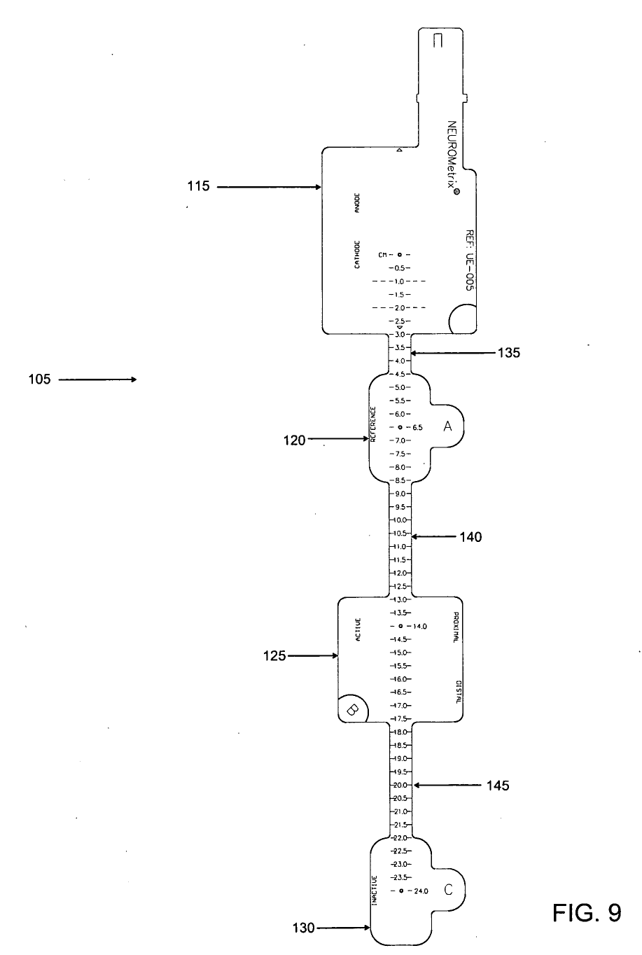

As seen in FIG. 9, the front side of substrate 110 is preferably labeled so as to show the location of the various elements. This labeling helps the healthcare provider avoid errors when applying the electrodes to the patient. Also printed on the front side of substrate 110 is a measurement scale to be used by the healthcare provider in accurately determining the distance between the stimulation site and a detection site when performing a sensory nerve assessment.

It should be appreciated that traces T are disposed on substrate 110 so as to be closely spaced and of short length, whereby to reduce the electrical noise effects of external EMI. This is because the short traces and small cross-sectional areas between the traces reduce induced electromagnetic noise signals.

### One Preferred Construction of the Multi-Electrode Assembly

The multi-electrode assembly of the present invention preferably comprises a flexible substrate having a conductive pattern deposited thereon, e.g., by silk screening, chemical plating or other conventional means well known to those skilled in the art.

More particularly, substrate 10, 110 may comprise a clear or colored MYLAR®. Where MYLAR® is used for substrate 10, 110, it may be in the range of 0.002 inches to 0.007 inches thick, depending on the desired stiffness. As noted above, it is desired that the islands (e.g., 15, 20, 25, 30, 35, 40 and/or 115, 120, 125, 130) have a flexible quality with a modest degree of stiffness so as to permit the islands to be easily manipulated by hand, and it is desired that the bridges (e.g., 45, 50, 55, 60, 65 and/or 135, 140, 145) have a highly flexible quality so as to permit the electrodes to be positioned in substantially any manner which may be desired by the healthcare provider. Graphical and textual information is preferably printed on the MYLAR® substrate (see FIGS. 2 and 9) to help guide the healthcare professional with proper placement. Temperature sensors 83, 175, and/or an electronic serial number memory component (not shown) are (to the extent that they are provided) attached to the MYLAR® substrate 10, 110 in electrical contact with the conductive pattern which forms traces T, e.g., with conductive epoxy. Temperature sensors 83, 175 are commonly available electronic components whose electrical values change with temperature. The aforementioned electronic serial number memory component is also a readily available programmable electronic component that is well known to those skilled in the art.

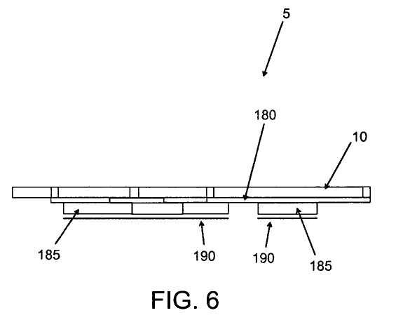

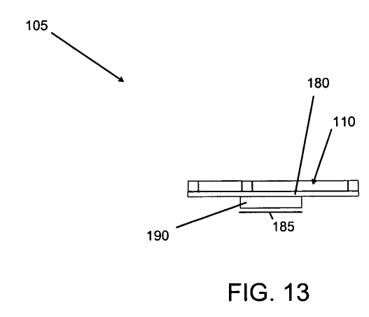

The conductive pattern also forms the various electrodes (e.g., stimulation electrodes 70 and 85, active detection electrode 75, inactive detection electrode 80, first active detection electrode 90, second active detection electrode 95, reference electrode 100, and/or stimulation electrodes 150, first active detection electrode 155, second active detection electrode 160, inactive detection electrode 165, reference electrode 170). These electrodes contact the skin to stimulate and detect the neuromuscular signals. A layer of polyethylene foam 180 (FIGS. 6 and 13), preferably in the range of 0.030 to 0.060 inches thick, with adhesive applied to one or both sides, is selectively die-cut or laser-cut to the desired shape, and then selectively laminated to the MYLAR® substrate 10, 110. Polyethylene foam 180 is used to form wells for holding the conductive gel which electrically couples the various electrodes to the skin.

A conductive gel layer 185 (FIGS. 6 and 13) is silk screened or dispensed over the electrode areas. The conductive gel is of a type available from Amgel or other suppliers well known to those skilled in the art, that has the property of remaining sticky and conductive after multiple applications to different areas of the body to test different nerves. During use, these areas contact and conform to the skin of the subject to acquire the myoelectrical signal. Protective release liner 190 is applied over the gel areas. The serial number and other information (as desired) are programmed into the electronic memory, and the assembly is finalized after being sealed into a pouch.

### Additional Aspects of the Present Invention

The present invention provides a novel approach for evaluating neuromuscular physiology. A multi-electrode assembly is described for significantly improving measurement flexibility for the healthcare provider in a cost-effective way. This is accomplished by using a multi-electrode assembly that has a single connection to the testing electronics but still permits the measurement of different locations on the body and different nerves, with the same multi-electrode assembly, by repositioning the electrode areas on the body. The apparatus of the present invention reduces time-consuming errors caused by multiple interconnects of individually-placed electrodes by utilizing a single connector to the testing electronics. Because of the printed information on the front surface of the multi-electrode assembly, the function of each of the electrode areas is clear to the healthcare provider, thereby reducing electrode placement errors. The printed traces on the substrate of the electrodes being in close proximity to each other, and of relatively short length, reduce the effects of induced signal noise from external EMI.

### Modifications

While the present invention has been described in terms of certain exemplary preferred embodiments, it will be readily understood and appreciated by those skilled in the art that it is not so limited, and that many additions, deletions and modifications may be made to the preferred embodiments discussed herein without departing from the scope of the invention.

What is claimed is:

1. A multi-electrode assembly for use in neuromuscular testing, the multi-electrode assembly comprising:
a substrate;
at least one stimulation electrode carried by the substrate;
at least one detection electrode carried by the substrate;
a connector carried by the substrate, the connector being configured for connection with testing electronics; and
a plurality of traces for electrically connecting the at least one stimulation electrode and the at least one detection electrode to the connector;
wherein the substrate is flexible so that the at least one stimulation electrode and the at least one detection electrode are independently positionable on a patient.

2. A multi-electrode assembly according to claim 1 wherein the substrate comprises a first island, a second island and a bridge connecting the first island to the second island, and further wherein the at least one stimulation electrode is carried by the first island, the at least one detection electrode is carried by the second island, and further wherein the bridge is flexible so that the first island and the second island are independently positionable on a patient.

3. A multi-electrode assembly according to claim 1 further comprising a temperature sensor carried by the substrate, wherein the temperature sensor is connected to the connector by the plurality of traces.

4. A method for conducting neuromuscular testing on a patient, the method comprising:
providing a multi-electrode assembly comprising:
a substrate;
at least one stimulation electrode carried by the substrate;
at least one detection electrode carried by the substrate;
a connector carried by the substrate, the connector being configured for connection with testing electronics; and
a plurality of traces for electrically connecting the at least one stimulation electrode and the at least one detection electrode to the connector;
wherein the substrate is flexible so that the at least one stimulation electrode and the at least one detection electrode are independently positionable on a patient;
positioning the multi-electrode assembly on a patient;
connecting the connector to testing electronics; and
performing a neuromuscular test on the patient.

5. A method according to claim 4 wherein performing a neuromuscular test on the patient comprises a motor nerve test.

6. A method according to claim 4 wherein performing a neuromuscular test on the patient comprises a sensory nerve test.

7. A method according to claim 4 further comprising, after performing the neuromuscular test on the patient, re-positioning the multi-electrode assembly on the patient and performing a further neuromuscular test on the patient.

8. A multi-electrode assembly for use in neuromuscular testing, the multi-electrode assembly comprising:
a substrate comprising first, second, third, fourth, fifth and sixth islands, and first, second, third, fourth and fifth bridges for connecting the first and second islands, the second and third islands, the first and fourth islands, the fourth and fifth islands and the fifth and sixth islands, respectively;
a first stimulation electrode carried by the first island, and a second stimulation electrode carried by the first island;
a first active detection electrode carried by the second island;
an inactive detection electrode carried by the third island;
a reference electrode carried by the fourth island;
a second active detection electrode carried by the fifth island;
a third active detection electrode carried by the sixth island;
a connector carried by the substrate, the connector being configured for connection with testing electronics; and
a plurality of traces for electrically connecting the first and second stimulation electrodes, first active detection electrode, inactive detection electrode, a reference electrode, third active detection electrode and fourth active detection electrode to the connector;
wherein the substrate is flexible so that the first and second stimulation electrodes, first active detection electrode, inactive detection electrode, reference electrode, third active detection electrode and fourth active detection electrode are independently positionable on a patient.

9. A method for conducting neuromuscular testing on a patient, the method comprising:
providing a multi-electrode assembly comprising:
a substrate comprising first, second, third, fourth, fifth and sixth islands, and first, second, third, fourth and fifth bridges for connecting the first and second islands, the second and third islands, the first and fourth islands, the fourth and fifth islands and the fifth and sixth islands, respectively;
a first stimulation electrode carried by the first island, and a second stimulation electrode carried by the first island;
a first active detection electrode carried by the second island;
an inactive detection electrode carried by the third island;
a reference electrode carried by the fourth island;
a second active detection electrode carried by the fifth island;
a third active detection electrode carried by the sixth island;
a connector carried by the substrate, the connector being configured for connection with testing electronics; and
a plurality of traces for electrically connecting the first and second stimulation electrodes, first active detection electrode, inactive detection electrode, a reference electrode, third active detection electrode and fourth active detection electrode to the connector;
wherein the substrate is flexible so that the first and second stimulation electrodes, first active detection electrode, inactive detection electrode, reference electrode, third active detection electrode and fourth active detection electrode are independently positionable on a patient;
positioning the multi-electrode assembly on a patient;
connecting the connector to testing electronics; and
performing a neuromuscular test on the patient.

10. A method according to claim 9 wherein performing a neuromuscular test on the patient comprises a motor nerve test.

11. A method according to claim 9 wherein performing a neuromuscular test on the patient comprises a sensory nerve test.

12. A method according to claim 9 wherein performing a neuromuscular test on the patient comprises both a motor nerve test and a sensory nerve test.

13. A method according to claim 9 wherein the motor nerve test and the sensory nerve test are performed one after the other.

14. A method according to claim 13 wherein the motor nerve test is performed before the sensory nerve test.

15. A method according to claim 13 wherein the motor nerve test is performed after the sensory nerve test.

16. A method according to claim 9 wherein the motor nerve test and the sensory nerve test are performed substantially simultaneously.

17. A method according to claim 9 further comprising, after performing the neuromuscular test on the patient, re-positioning the multi-electrode assembly on the patient and performing a further neuromuscular test on the patient.

18. A multi-electrode assembly for use in neuromuscular testing, the multi-electrode assembly comprising:
a substrate comprising first, second, third and fourth islands, and first, second and third bridges for connecting the first and second islands, the second and third islands and the third and fourth islands, respectively;
a first stimulation electrode carried by the first island;
a first active detection electrode carried by the third island;
an inactive detection electrode carried on the fourth island;
a second active detection electrode carried by the third island;
a reference electrode carried by the second island;
a connector carried by the substrate, the connector being configured for connection with testing electronics; and
a plurality of traces for electrically connecting the first stimulation electrode, first active detection electrode, inactive detection electrode, second active detection electrode and reference electrode to the connector;
wherein the substrate is flexible so that the first stimulation electrode, first active detection electrode, inactive detection electrode, second active detection electrode and reference electrode are independently positionable on a patient.

19. A method for conducting neuromuscular testing on a patient, the method comprising:
providing a multi-electrode assembly comprising:
a substrate comprising first, second, third and fourth islands, and first, second and third bridges for connecting the first and second islands, the second and third islands and the third and fourth islands, respectively;
a first stimulation electrode carried by the first island;
a first active detection electrode carried by the third island;
an inactive detection electrode carried on the fourth island;
a second active detection electrode carried by the third island;
a reference electrode carried by the second island;
a connector carried by the substrate, the connector being configured for connection with testing electronics; and
a plurality of traces for electrically connecting the first stimulation electrode, first active detection electrode, inactive detection electrode, second active detection electrode and reference electrode to the connector;
wherein the substrate is flexible so that the first stimulation electrode, first active detection electrode, inactive detection electrode, second active detection electrode and reference electrode are independently positionable on a patient;
positioning the multi-electrode assembly on a patient;
connecting the connector to testing electronics; and
performing a neuromuscular test on the patient.

20. A method according to claim 19 wherein performing a neuromuscular test on the patient comprises a motor nerve test.

21. A method according to claim 19 wherein performing a neuromuscular test on the patient comprises a sensory nerve test.

22. A method according to claim 19 further comprising, after performing the neuromuscular test on the patient, re-positioning the multi-electrode assembly on the patient and performing a further neuromuscular test on the patient.
